(19) United States
(12) Patent Application Publication
(10) Pub. No.: US 2021/0236528 A1
(43) Pub. Date: Aug. 5, 2021

(54) COMPOSITION COMPRISING ISOMERS OF INOSITOL AND ITS USE

(71) Applicant: SOCIETE DES PRODUITS NESTLE S.A., Vevey (CH)

(72) Inventors: Irma Silva Zolezzi, Singapore (SG); Jean-Philippe Godin, Ecublens (CH); Shiao-Yng Chan, Singapore (SG); Keith Malcolm Godfrey, Hampshire (GB)

(21) Appl. No.: 17/269,660

(22) PCT Filed: Aug. 22, 2019
(86) PCT No.: PCT/EP2019/072418

§ 371 Date: Feb. 19, 2021

(51) Int. Cl. A61K 31/7004; A61K 31/59; A61K 31/675; A61K 31/714; A61K 31/525; A61K 33/30; A61K 33/26; A61P 3/10; A61K 35/747; A61K 35/745; A61K 9/00; A23L 33/00; A23L 33/125; A23L 33/135; A23L 33/155; A23L 33/16; A23L 2/52; A23K 20/163; A23K 10/18; A23K 20/174; A23K 20/20; A23K 50/40

(52) U.S. Cl. CPC A61K 31/7004; A23Y 2300/49; A61K 31/675; A61K 31/714; A61K 31/525; A61K 33/30; A61K 33/26; A61P 3/10; A61K 35/747; A61K 35/745; A61K 9/0056; A61K 9/0095; A23L 33/40; A23L 33/125; A23L 33/135; A23L 33/155; A23L 33/16; A23L 2/52; A23K 20/163; A23K 10/18; A23K 20/174; A23K 20/30; A23K 50/40; A23V 2002/00; A23Y 2220/73; A61K 31/59

(57) ABSTRACT

A combination of scyllo-inositol and myo-inositol, wherein, the scyllo-inositol and myo-inositol are nutritional ingredients for use in the prevention or treatment of a condition selected from the group consisting of prediabetes, type II diabetes, gestational diabetes mellitus and a condition associated with any of the forgoing in a subject, and a composition comprising scyllo-inositol and myo-inositol.

### FIELD OF THE INVENTION

The invention relates to a combination of scyllo-inositol and myo-inositol for use in the prevention or treatment of insulin resistance, prediabetes, type II diabetes and/or Gestational Diabetes mellitus (GDM) in a subject. The composition further relates to a composition comprising scyllo-inositol and myo-inositol.

### BACKGROUND

The human body prevents elevated blood glucose levels by secreting insulin, thus triggering the absorption of glucose from the blood by insulin-sensitive tissue e.g. liver, muscle and adipose tissue. However, not everyone responds adequately to insulin. Such people are said to be insulin resistant and to suffer from prediabetes. When a person has prediabetes the human body compensates for the inadequate response to insulin by secreting more of it, however, eventually the body can fail to keep up with the increased demand and type II diabetes can ensue.

According to the World Health Organization, at least 171 million people worldwide suffer from type II diabetes and it is been estimated that by 2030 this number will have almost doubled. Prediabetes (also referred to as impaired glucose tolerance) is even more common and it is estimated that, in the United States alone, more than one out of three adults suffer from this. Prediabetes and type II diabetes are associated with an increased risk of a variety of diseases including cardio vascular disease—one of the leading causes of death in both the developed and developing world.

An impaired glucose tolerance can occur during pregnancy and can result in gestational diabetes mellitus. This condition can increase the risk of a number of maternal-fetal conditions, including macrosomia and premature birth.

Dietary and lifestyle changes, including healthier dietary habits and increased exercise, can be very efficient in preventing or treating prediabetes, type II diabetes and/or gestational diabetes mellitus, however, patient compliance can be problematic. Drugs such as biguanides and thiazolidinediones may also be used. However, many of these have unwanted side effects. Moreover, there is no practice of giving such drugs to prevent prediabetes and many are unsuitable for use during pregnancy.

Accordingly, there is a need to find alternative ways to treat or prevent prediabetes, type II diabetes, gestational diabetes mellitus and/or a condition associated with any of the foregoing in a subject. In particular there is a need to find ways that may not suffer from one or more of the drawbacks of the prior art. An object of the present invention is to address this need.

### SUMMARY OF THE INVENTION

The invention is set out in the claims and in the detailed description included herein. Described is a combination of scyllo-inositol and myo-inositol for use in the prevention or treatment of a condition selected from the group consisting of prediabetes, type II diabetes, gestational diabetes mellitus and a condition associated with any of the forgoing in a subject wherein, the scyllo-inositol and myo-inositol are nutritional ingredients. Also described are composition comprising scyllo-inositol and myo-inositol wherein, said composition is a product selected from the group consisting of: a nutritional product, a food product, a functional food product, a healthy ageing product, a dairy product, a nutritional supplement, a pharmaceutical formulation, a beverage product, a diet product, and a pet food product.

### DRAWINGS

FIG. 1—A scheme showing one way to synthesize scyllo-inositol from myo-inositol via the orthoformate, wherein the reaction condition are a) NaH, BzCl, DMF, RT; b) tosyl chloride, pyridine, 80-100° C.; c)isobutylamine, MeOH, reflux; d) (COCl)₂, DMSO, CH₂CH₂, −78° C., then Et₃N, RT; e) NaBH₄, MeOH/THF (4:1), RT; f) NaOMe, reflux; g) TFA/water 4:1.

FIG. 2—spider graph showing the relative plasma concentrations of inositol isomers measured in humans having different glucose tolerance status.

FIG. 3 spider graph showing the relative urine concentrations of inositol isomers measured in humans having different glucose tolerance status.

FIG. 4a—Spider graph showing the relative urinary inositol levels in Caucasian women (N=90) by glucose tolerance group.

FIG. 4b—Spider graph showing the relative plasma inositol levels in Caucasian women (N=90) by glucose tolerance group

FIG. 5—Spider graph showing the relative plasma inositol levels in Asian women (N=144) by glucose tolerance group

FIG. 6—Spider graph showing the relative urinary inositol levels in Asian women (N=144) by glucose tolerance group

FIG. 7. Chart showing the scyllo-inositol level in plasma by glucose tolerance status for 249 women (All), for Asian women (N=144) and Caucasian women (N=90)

FIG. 8. Chart showing the myo-inositol level in plasma by glucose tolerance status for 249 women (All), for Asian women (N=144) and Caucasian women (N=90)

FIG. 9. Chart showing the Myo to scyllo-inositol ratio in plasma by glucose tolerance status for 249 women (All), for Asian women (N=144) and Caucasian women (N=90)

### DETAILED DESCRIPTION

The inventors have surprisingly found that, in comparison to subjects having normal glucose tolerance, the plasma concentration of both myo-inositol to scyllo-inositol may be lower in subjects suffering with an impaired glucose tolerance or type II diabetes.

Without wishing to be bound by theory, the inventors believe that myo-inositol and scyllo-inositol may play vital yet independent roles in glucose management and that an adequate intake and physiological concentration of both of these inositol isomers may be necessary to avoid any impaired glucose tolerance. Accordingly, administering a combination of scyllo-inositol and myo-inositol to a subject may prevent or treat prediabetes, type II diabetes, gestational diabetes mellitus or a condition associated with any of the forgoing in a subject.

In a first aspect of the present invention there is provided a combination of scyllo-inositol and myo-inositol for use in the prevention or treatment of a condition selected from the group consisting of prediabetes, type II diabetes, gestational diabetes mellitus and a condition associated with any of the forgoing in a subject wherein, the scyllo-inositol and myo-inositol are nutritional ingredients.

The term “treat” as used herein encompasses amelioration and/or alleviation of a condition i.e. the amelioration and/or alleviation of the symptoms of a condition. It may for example encompass the reduction of the severity of a condition in a subject.

The term “prevent” as used herein refers to the prevention of the occurrence, or reduction of the risk of the occurrence, of a condition in a subject.

The term “Nutritional ingredients” as used herein refers to ingredients that are not marketed as pharmaceuticals and that have a nutritional value e.g. may contribute to a daily calorie intake.

The term “subject” as used herein refers to a mammal and more particularly a cat, a dog or a human. The human may be an adult, child or infant. The human may be a woman for example a woman who is trying to get pregnant, who is pregnant, or who is lactating. The subject may also be the offspring of a woman who is trying to get pregnant, who is pregnant, or who is lactating.

In an embodiment of the invention the subject is a mammal selected from the group consisting of a cat, a dog and, a human.

The term scyllo-inositol as used herein refers (1r,2r,3r,4r,5r,6r)-Cyclohexane-1,2,3,4,5,6-hexol.

The term myo-inositol as used herein refers to cis-1,2,3,5-trans-4,6cyclohexanehexol.

In addition to that detailed above, the inventors have also found that the ratio of the concentration of myo-inositol to scyllo-inositol in both plasma and urine may be lower in subjects having a normal glucose tolerance in comparison to those having an impaired glucose tolerance or type II diabetes.

Without wishing to be bound by theory, the inventors believe that having an adequate physiological balance i.e. ratio, between scyllo-inositol and myo-inositol, may be particularly effective at preventing an impaired glucose tolerance, and that subjects at risk of developing or suffering from prediabetes, type II diabetes or gestational diabetes mellitus may have an insufficient dietary intake of these compounds and/or, that epimerisation activities involving the conversion of myo-inositol to scyllo-inositol may be impaired. Accordingly, administering a combination of scyllo-inositol and myo-inositol in a specific ratio, designed to promote an adequate balance of these inositol isomers, to a subject, may prevent or treat prediabetes, type II diabetes, gestational diabetes mellitus or a condition associated with any of the forgoing in a subject.

In particular the inventors have found that subjects that do not have prediabetes or type II diabetes have a lower myo-inositol to scyllo-inositol ratio and in particular may have a plasma myo-insoitol to scyllo-inositol ratio of less than 80:1, for example less than 60:1, less than 55:1, less than 46:1, less than 35:1. Accordingly, it may be beneficial if these compounds are administered in a ratio falling within one of these ranges.

In an embodiment of the present invention the myo-inositol and scyllo-inositol are administered in a weight ratio of myo-inositol to scyllo-inositol in the range of 1:1 to 80:1 for example 1:1 to 55:1 for example 1:1 to 46:1, for example 1:1 to 35:1.

Non limiting examples of ratios of less than 55:1 include 54:1, 53:2, 52:1, 51:1, 50;1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1.

Further to the above, the inventors found that subjects that do not have prediabetes or type II diabetes and who also have a favourable metabolic profile may have a plasma myo-insoitol to scyllo-inositol ratio of less than 55:1, less than 46:1 or less than 35 to 1. Accordingly, it may be particularly beneficial if these compounds are administered in a ratio falling within one of these ranges.

The myo-inositol and scyllo-inositol may for example be administered in a weight ratio in the range of 1:1 to 55:1 for example 5:1 to 46:1, 8:1 to 35:1.

In an embodiment of the present invention the myo-inositol and scyllo-inositol are administered in a weight ratio of myo-inositol to scyllo-inositol in the range of 1:1 to 55:1.

In the context of the present invention, a human subject is considered to have a favourable metabolic profile if they meet the following criteria:
their BMI is less than 25 if they are Caucasian and less than 23 if they are Asian,
their waist measurement is less than or equal to 80 cm,
their waist to hip ration is less than 85 and,
they have a HOMA2-IR of less than 1.21.

Numerous conditions are associated with prediabetes, type II diabetes and/or gestational diabetes.

In an embodiment of the invention the condition associated with prediabetes or type II diabetes is selected from the group consisting of cardio vascular disease, stroke, circulatory problems, diabetic retinopathy, kidney failure, hearing loss, fatty liver disease, an impaired cognitive ability, polycystic ovary syndrome and metabolic syndrome.

In an embodiment of the invention the condition associated with gestational diabetes mellitus is selected from the group consisting of preterm and caesarean delivery, birth injury to the mother or baby, shoulder dystocia, macrosomia, excessive offspring blood glucose concentration, excess weight/adiposity and associated metabolic disorders e.g. type II diabetes, fatty liver disease and obesity immediately after birth and later in the life of the offspring, and an increased risk for the mother of having or developing type 2 diabetes immediately after birth and later in life.

Scyllo-inositol and myo-inositol may be particularly effective at preventing and/or treating prediabetes, type II diabetes and gestational diabetes mellitus in subject's who are at risk of suffering from one or more of these conditions.

Accordingly, in an embodiment of the invention the subject e.g. the human subject, is at risk of suffering from pre-diabetes, type II diabetes or gestational diabetes mellitus e.g. because they do not have a favourable metabolic profile and/or have a history or family history of one or more of these conditions.

Gestational diabetes mellitus is a condition that affects pregnant woman. Accordingly, in another embodiment of the present invention the condition is gestational diabetes mellitus or a condition associated therewith and the subject is a woman who is trying to get pregnant, is pregnant or who is lactating or is her offspring.

If the scyllo-inositol and myo-inositol are administered to a human subject desiring to get pregnant, they may be administered during at least 1, 2, 3 or 4 months preceding the pregnancy or desired pregnancy. If they are administered to a pregnant subject, they may be administered throughout or partially throughout the pregnancy e.g. for at least 4, at least 8, at least 12, at least 16, at least 20, at least 24, at least 28, or at least 36 weeks depending on the gestational period of the subject. Administration may also continue throughout or partially throughout the lactation period of said subject.

Since the risk of gestational diabetes mellitus increases in the second and third trimester of pregnancy, administration may be particularly beneficial in the second and third trimester of pregnancy for the prevention or treatment of GDM, or the prevention of a condition associated therewith in a pregnant subject or its offspring.

In an embodiment of the invention the scyllo-inositol and myo-inositol are administered in at least the second and/or third trimester of pregnancy wherein the subject is a pregnant woman or her offspring.

The scyllo-inositol and myo-insoitol may be administered to a subject in any effective amount. An effective amount may be any amount that improves, by any degree an impaired glucose tolerance in a subject.

It is well within the purview of the skilled person to determine an effective amount. An effective amount may, for example, be determined by testing the effect of an amount on a subject's fasting glucose plasma concentration, or fasting HbA1c concentration, or on glycemic levels at 1 hr, 2 hr during an OGTT. An effective amount should improve a subject's fasting glucose plasma concentration and/or HbA1c concentration, and/or lower a subject's glycemic response. In particular if the subject is suffering from prediabetes, type II diabetes or gestational diabetes mellitus.

An effective amount may also be determined by testing the effect of an amount on the number of subject's that go on to develop prediabetes, type II diabetes or gestational diabetes in a cohort.

Typically, an effective amount will depend on the type, age, size, health status, lifestyle and/or genetic heritage of the subject. The effective amount may be split into several smaller amounts and administered throughout the day so as the total daily intake is the effective amount.

Scyllo-inositol may for example be administered to a subject in an amount of up to 1 g, for example up to 0.5 g, up to 0.25 g, up to 0.15 g, up to 0.125 g, up to 0.1 g, up to 0.09 g, up to 0.08 g, up to 0.07 g, up 0.05 g per day (daily amount/dose).

In an embodiment of the invention the scyllo-inositol is administered to a subject in an amount up to 1 g per day.

The myo-inositol may for example be administered in an amount of 0.2 to 5 g, for example 1.5 to 5 g, 2 to 5 g or 2 to 4 g per day (daily amount/dose).

In an embodiment of the invention the myo-inositol is administered to a subject in an amount up to 5 g per day.

Scyllo-inositol and myo-inositol may be employed in the invention as scyllo-inositol and myo-inositol respectively or in their phosphate forms e.g. myo-inositol P1-6 and scyllo-inositol bis, tris or hexakisphosphate; in meso or racemic forms. Other derivatives of myo-inositol or scyllo-inositol could also be employed for example fluorinates, C-methyl and, deoxy-scyllo-inositols.

Ordinarily scyllo-insoitol and myo-inositol will be employed.

In an embodiment of the invention the myo-inositol and scyllo-insoitol are administered as scyllo-inositol and myo-inositol respectively.

Myo-inositol and scyllo-inositol are commercially available, for example from sigma-Aldrich or other ingredient suppliers.

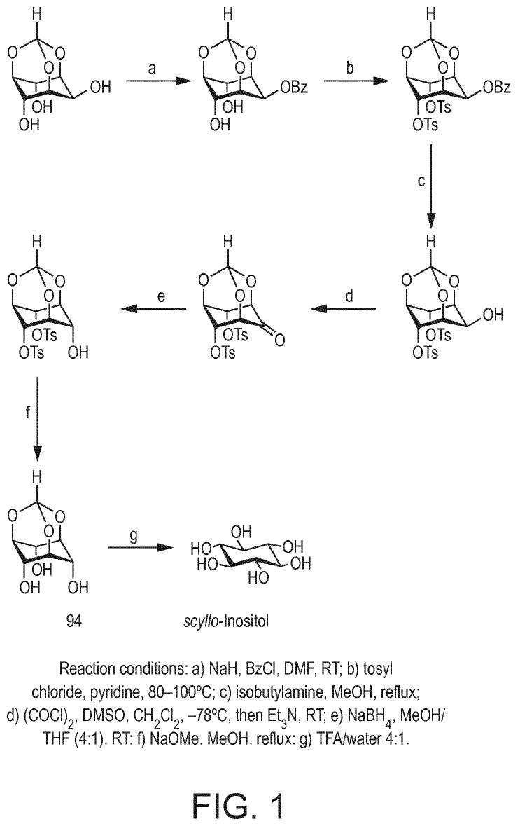

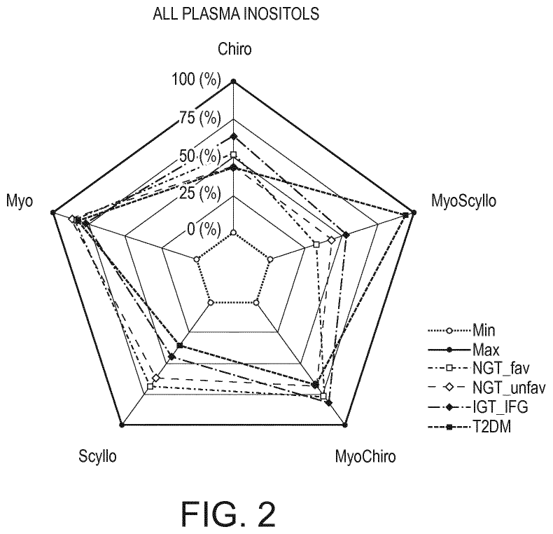

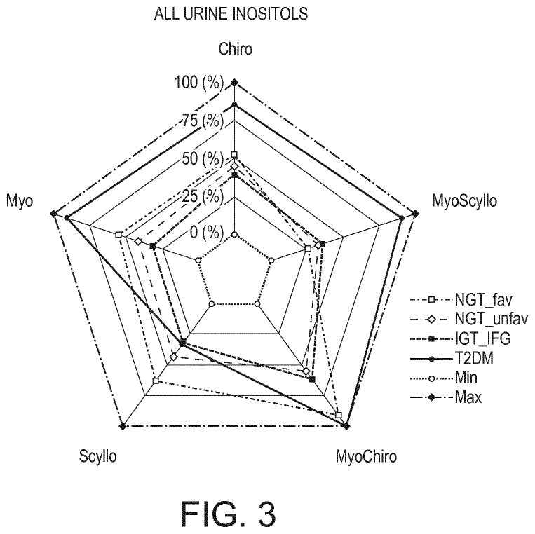

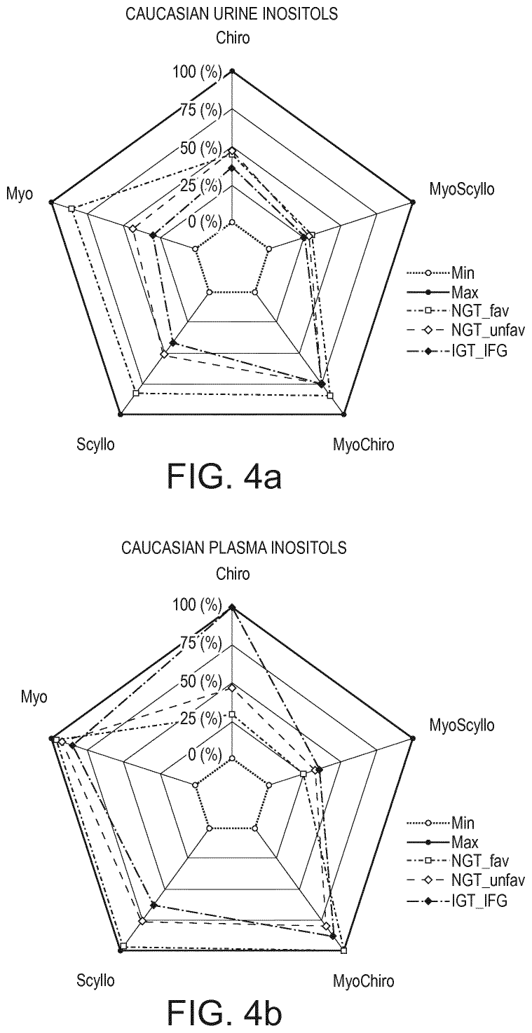

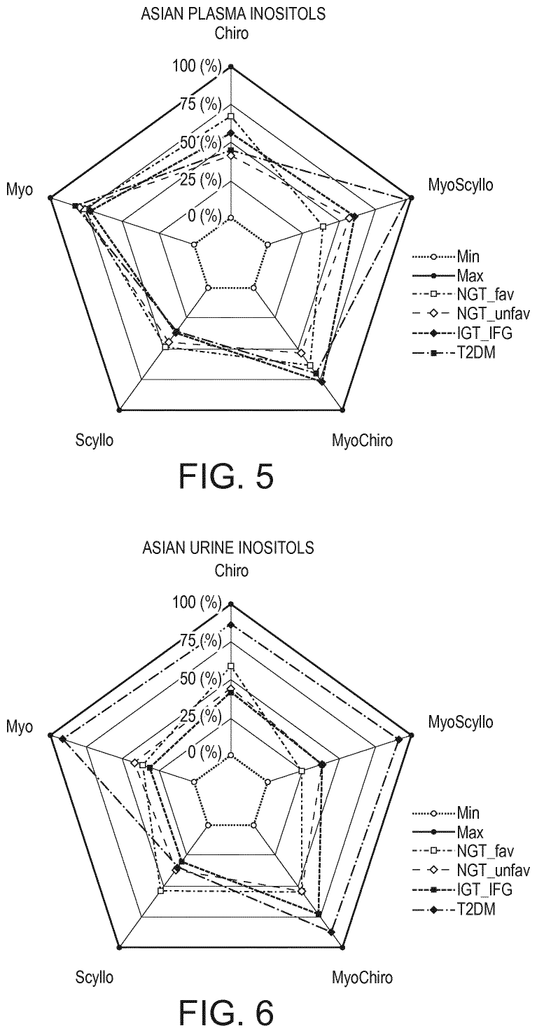

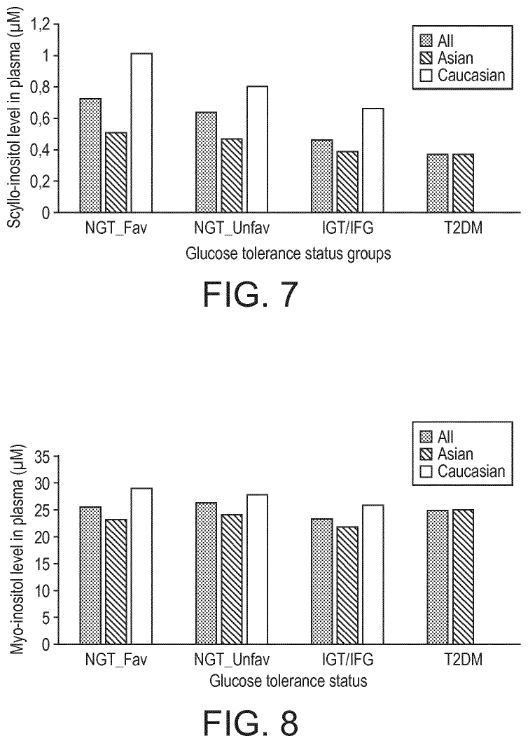

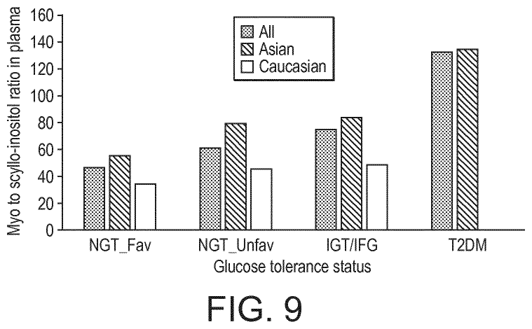

Scyllo-inositol may be produced via a variety of methods, for example scyllo-inositol may be produced from myo-inositol by the process set out in the scheme of FIG. 1. Methods for manufacturing scyllo-inostitol are further set out in Thomas et al. (2016), The other “inositols” and their phosphates: synthesis, biology, and medicine (with recent advances in myo-inositol chemistry, Angew. Chem. Int. Ed. 55, 16-14-1650 which is hereby incorporated by reference in its entirity.

Alternatively, scyllo-inositol may be produced from myo-inositol in a bio-conversion process using microorganisims such as Pseudomos and Acetobacter. For example, scyllo-inosose may be produced from myo-inositol in a bio-conversion process using micro-organisims belonging to the genus Acetobacter and, the produced Scyllo-inosose may subsequently be enzymatically reduced to scyllo-inositol.

Myo-inositol and/or scyllo-inositol may also be present in plant or fruit concentrate e.g. coconut, grains such as buckwheat, and citrus. The myo-inositol or scyllo-inositol may be extracted from these fruit or my be employed in the form of these ingredients. The skilled person would be able to calculate the amount of myo-inositol and/or scyllo-inositol delivered by such extracts/ingredients on the basis of the concentration of myo-inositol and/or scyllo-inositol in said extract/ingredient and the amount of the extract/ingredient employed.

The scyllo-inositol and myo-inositol may be administered simultaneously, sequentially or separately.

The myo-inositol and scyllo-inositol may be administered in combination with other ingredients e.g. functional ingredients (ingredients known to give rise to a function or beneficial effect).

Probiotics have been found to improve the gut barrier function and to help nutrients pass through the gut. Administrating probiotics in combination with the combination of scyllo-inositol and myo-inositol may therefore enhance the absorption of these compounds.

Accordingly, in an embodiment the scyllo-inositol and myo-inositol are administered in combination with a probiotic selected from the group consisting of Lactobacillus Rhamnosus GG (CGMCC 1.3724) also known as LPR, Bifidobacterium Lactus BB-12 (CNCM I-3446) also known as BL818, and a combination thereof.

Lactobacillus Rhamnosus GG (CGMCC 1.3724) is commercially available for example from Valio Oy.

Bifidobacterium Lactus BB-12 (CNCM I-3446) is commercially available for example from Christian Hansen.

The term probiotic as used herein refers to live probiotic bacteria, non-replicating probiotic bacteria, dead probiotic bacteria, non-viable probiotic bacteria, fragments of probiotic bacteria such as DNA, metabolites of probiotic bacteria, cytoplasmic compounds of probiotic bacteria, cell wall materials of probiotic bacteria, culture supernatants of probiotic bacteria, and combinations of any of the foregoing. The probiotic may for example be live probiotic bacteria, non-replicating probiotic bacteria, dead probiotic bacteria, non-viable probiotic bacteria, or any combination thereof.

In an embodiment of the invention the probiotic is live probiotic bacteria.

Additional vitamins and minerals may also be administered in combination with the scyllo-inositol and myo-inositol. For example, the composition may contain one or more of the following micronutrients, calcium, magnesium, phosphorus, iron, zinc, copper, iodine, selenium, vitamin A or retinol activity equivalent (RAE) for example in the form of beta carotene or a mix of carotenoids, Vitamin C, Vitamin B1, niacin, folic acid, biotin, Vitamin E, vitamin B2, vitamin B6, vitamin B15, vitamin D, iron, zinc.

Vitamins and minerals may be administered in amounts in accordance with the recommendations of Government bodies such as the USRDA. For example: 100 to 2500 mg calcium, 35 to 350 mg magnesium, 70 to 3500 mg phosphorus, 2.7 to 45 mg iron, 1.1 to 40 mg zinc, 0.1 to 10 mg copper, 22 to 1,100 μg iodine, 6 to 400 μg selenium, 77 to 3000 μg of vitamin A or retinol activity equivalent (RAE) for example in the form of beta carotene or a mix of carotenoids, 8.5 to 850 mg Vitamin C, 0.14 to 14 mg Vitamin B1, 1.8 to 35 mg niacin, 60 to 1000 μg folic acid, 3 to 300 μg biotin, 1.9 to 109 μg Vitamin E.

In an embodiment of the invention, the scyllo-inositol and myo-inositol are administered in combination with an ingredient selected from the group consisting of: vitamin B2, vitamin B6, vitamin B12, vitamin D, iron, zinc and a combination of any of the foregoing.

Vitamin B2 may for example be administered in an amount from 0.14 to 14 mg per day (daily dose). Vitamin B6 may for example be administered in an amount from 0.19 to 19 mg per day (daily dose). Vitamin B12 may for example be administered in an amount from 0.26 to 26 μg per day (daily dose). Vitamin D may for example be administered in an amount from 1.5 to 100 μg per day (daily dose), Zinc may for example be administered in an amount of from 1.1 to 40 mg per day (daily dose).

In a specific embodiment of the invention, the scyllo-inositol and myo-inositol is administered in combination with vitamin B2, vitamin B6, Vitamin B12 and vitamin D wherein said ingredients are administered in amounts equating to 1.8 mg of vitamin B2, 2.6 mg of vitamin B6, 5.2 μg of vitamin B12 and 10 μg of vitamin D per day (daily dose).

In yet another embodiment the scyllo-inositol and myo-inositol may be administered in combination with D chiro-inositol. D chiro-inositol is known to have blood glucose lowering properties.

The combination of scyllo-inositol and myo-inositol may be administered in the form of a composition, said composition may be a product selected from the group consisting of: a nutritional product, a food product, a functional food product, a healthy ageing product, a dairy product, a nutritional supplement, a beverage product, a diet product, and a pet food product.

In an embodiment of the present invention the scyllo-inositol and myo-inositol are administered in the form of a composition wherein, said composition is a product selected from the group consisting of: a nutritional product, a food product, a functional food product, a healthy ageing product, a dairy product, a nutritional supplement, a beverage product, a diet product, and a pet food product.

In an embodiment said composition comprises scyllo-inositol and myo-inositol in a weight ratio of less than 80:1, for example less than 55:1, less than 35:1.

The composition may for example comprise myo-inositol and myo-inositol in a weight ratio range of 1:1 to 55:1 for example 5:1 to 46:1, 8:1 to 35:1.

The composition may comprise any other ingredient for example one or more ingredients set out herein e.g. probiotics vitamins and minerals.

For example, in an embodiment the composition may comprise myo-inositol, scyllo-inositol, Lactobacillus rhamnosus GG (CGMCC 1.3724) and/or Bifidobacterium lactus BB-12 (CNCM I-3446).

For example, in an embodiment the composition may comprise myo-inositol, scyllo-inositol, Lactobacillus rhamnosus GG (CGMCC 1.3724) and/or Bifidobacterium lactus BB-12 (CNCM I-3446) and an ingredient selected from the group consisting of: one or more of vitamin B2, vitamin B6, vitamin B12, vitamin D, iron, zinc and a combination of any of the foregoing.

The composition may also comprise other ingredients commonly used in the form of composition in which it is employed e.g powdered nutritional supplement, a food product, or a dairy product. Non limiting examples of such ingredients include: other nutrients, for instance, selected from the group of lipids (optionally in addition to DHA and ARA), carbohydrates, and protein, micronutrients (in addition to those set out above), conventional food additives such as anti-oxidants, stabilizers, emulsifiers, acidulants, thickeners, buffers or agents for pH adjustment, chelating agents, colorants, excipients, flavor agents, osmotic agents, preservatives, sugars, sweeteners, texturizers, emulsifiers, water and any combination thereof.

The term “food product”, as used herein, refers to any kind of product that may be safely consumed by a human or animal. Said food product may be in solid, semi-solid or liquid form and may comprise one or more nutrients, foods or nutritional supplements. For instance, the food product may additional comprise the following nutrients and micronutrients: a source of proteins, a source of lipids, a source of carbohydrates, vitamins and minerals. The composition may also contain anti-oxidants, stabilizers (when provided in solid form) or emulsifiers (when provided in liquid form).

The term “functional food product” as used herein, refers to a food product providing a additional health-promoting or disease-preventing function to the individual.

The term “dairy products”, as used herein, refers to food products produced from animals such as cows, goats, sheep, yaks, horses, camels, and other mammals. Examples of dairy products are lowfat milk (e.g. 0.1%, 0.5% or 1.5% fat), fat-free milk, milk powder, whole milk, whole milk products, butter, buttermilk, buttermilk products, skim milk, skim milk products, high milk-fat products, 15 condensed milk, crème fraiche, cheese, ice cream and confectionery products, probiotic drinks or probiotic yoghurt type drinks.

The term “beverage product” as used herein, refers to a nutritional product in liquid or semi-liquid form that may be safely consumed by an individual.

The term “pet food product” as used herein refers to a nutritional product that is intended for consumption by pets. A pet, or companion animal, as referenced herein, is to be understood as an animal selected from dogs, cats, birds, fish, rodents such as mice, rats

The term “nutritional supplement” as used herein, refers to a nutritional product that provides nutrients to an individual that may otherwise not be consumed in sufficient quantities by said individual. For instance, a nutritional supplement may include vitamins, minerals, fiber, fatty acids, or amino acids. Nutritional supplements may for example be provided in the form of a pill, a tablet, a lozenger, a chewy capsule or tablet, a tablet or capsule, or a powder supplement that can for example be dissolved in water or sprinkled on food. Nutritional supplements typically provide selected nutrients while not representing a significant portion of the overall nutritional needs of a subject. Typically they do not represent more than 0.1%, 1%, 5%, 10% or 20% of the daily energy need of a subject. A nutritional supplement may be use during pregnancy e.g. a maternal supplement.

In an embodiment of the present invention the composition is for use in the treatment and/or prevention of gestational diabetes mellitus or a condition associated therewith, the composition is a maternal supplement and the subject is a woman who is trying to get pregnant, is pregnant or who is lactating or, is her offspring.

In another aspect of the present invention there is provided the use of scyllo-inositol and myo-inositol for use in the manufacture of a composition for use in the prevention and/or treatment of a condition selected from the group consisting of prediabetes, type II diabetes, gestational diabetes mellitus and a condition associated with any of the foregoing in a subject wherein, the scyllo-inositol and myo-inositol are nutritional ingredients.

In another aspect of the present invention there is provided a method of preventing and/or treating a condition selected from the group consisting of prediabetes, type II diabetes, gestational diabetes mellitus and a condition associated with any of the foregoing in a subject, wherein said method comprises the step of administering to said subject scyllo-inositol and myo-inositol wherein, the scyllo-inositol and myo-inositol are nutritional ingredients.

In addition to that set out above, the inventors have also found that the average myo-inositol and scyllo-inositol plasma concentration and, the average myo-inositol to syllo-inositol ratio measured in their study differed between Asian and Caucasian subjects not having prediabetes or type II diabetes. In particular, the inventors found that, in comparison to Caucasian subjects, Asian subjects may on average have a lower myo-inositol and/or scyllo-inositol plasma concentration and/or, a higher plasma myo-inositol to scyllo-inositol ratio.

Without wishing to be bound by theory the inventors believe that these differences may be because of physiological differences (different trajectories of glycaemia, insulin sensitivity and insulin secretion) between Caucasian and Asian subjects and, possibly indicative of the need to supplement Asian subjects with an increased amount of myo-inositol and/or scyllo-inositol and/or with a higher ratio of myo-inositol to scyllo-inositol, compared to Caucasian subjects.

Accordingly in another embodiment there is provided a product range comprising two products, wherein one product is formulated taking into consideration the specific needs of Asian subjects and wherein the other product is formulated taking into consideration the specific needs of Caucasian subjects and, wherein said product for formulated taking into consideration the specific needs of Asian subjects comprises more myo-inositol and/or more scyllo-inositol and/or a higher ratio of myo-inositol to scyllo-inositol, than the product formulated taking into consideration the specific needs of Caucasian subjects.

A product that is formulated taking into consideration the specific needs of an Asian subject may be marketed specifically for use in this sub-set of the population.

A product that is formulated taking into consideration the specific needs of a Caucasian subject may be marketed specifically for use in this sub-set of the population.

In an embodiment the product formulated taking into consideration the specific needs of an Asian subject comprises more myo-inositol than the product formulated taking into consideration the specific needs of a Caucasian subject.

In an embodiment the product formulated taking into consideration the specific needs of an Asian subject comprises more scyllo-inositol than the product formulated taking into consideration the specific needs of a Caucasian subject.

In an embodiment the product formulated taking into consideration the specific needs of an Asian subject comprises more myo-inositol and more scyllo-inositol than the product formulated taking into consideration the specific needs of a Caucasian subject.

In an embodiment the product formulated taking into consideration the specific needs of an Asian subject comprises myo-inositol and scyllo-inositol wherein the myo-inositol to scyllo-inositol ration is higher than the product formulated taking into consideration the specific needs of a Caucasian subject.

In an embodiment the product formulated taking into consideration the specific needs of an Asian subject comprises more myo-inositol and more scyllo-inositol and, the myo-inositol to scyllo-inositol ratio is higher than in the product formulated taking into consideration the specific needs of a Caucasian subject.

Those skilled in the art will understand that they can freely combine all features of the present invention disclosed herein. In particular, features described for different embodiments of the present invention may be combined. Where known equivalents exist to specific features, such equivalents are incorporated as if specifically referred to in this specification. Further advantages and features of the present invention are apparent from the figure and non-limiting example.

The term “and/or” used in the context of the “X and/or Y” should be interpreted as “X”, or “Y”, or “X and Y”.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 8, from 3 to 7, from 4 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

Unless defined otherwise, all technical and scientific terms used herein have the same meaning as commonly understood by one of ordinary skill in the art.

The present invention will now be described in further details by the way of the following examples.

### EXAMPLES

### Studies

Two studies were considered in this report.
1. The first one was a pilot study carried out Nestlé Research Center (NRC) in 2016 with healthy pregnant and non-pregnant women (in total N=12).
2. The second one was based on the withdraw women from another study on blood glucose management (N=249). The characteristics of the subjects from UK, SG and NZ enrolled in this exploratory study are described in Tables 1 and 2.

### Pilot Study 1

Twelve women from Nestlé Research Center (NRC, Lausanne, Switzerland) were recruited, corresponding to three groups: non-pregnant (N=3), pregnant in the 2nd trimester (N=3) and pregnant in the 3rd trimester (N=3). Exclusion criteria included chronic disease (metabolic, digestive, renal or cardiovascular) of clinical relevance according to the investigator, and lactating. The study was approved by the Swiss Ethics Committee. Per subject, a total of 20 mL of blood was collected in to EDTA-containing tubes and 50 mL urine were collected and frozen at −80° C. until analysis. No anthropometric and other metabolic parameters were recorded in this pilot study.

Withdrawn Subjects from Study 2—On Blood Glucose Management (N=249).

Only withdrawn women from this study 2 were included in this sub-study. Women were from UK (N=46), NZ (N=89), SG (N=117). All the data (anthropometric and biochemical parameters) were collected and measured. Plasma and urine inositol isomers were measured using a new validated UHPLC-MSMS method. Study 2 was approved by different Institutional Review Boards and the study design published (Godfrey K M et al, Trials. 2017 Mar. 20; 18(1):131.). Subjects were classified as normoglycemic (NGT), impaired glucose tolerance (IGT), and type 2 diabetic mellitus (T2DM) based upon the results of fasting glucose and the oral glucose tolerance test (OGTT). Subjects with 2-hours glucose were levels <7.8 mmol/L were considered as normal glucose tolerant (NGT), while those with 2-hour glucose between 7.8-11.1 mmol/L were deemed as having impaired glucose tolerance (IGT). Individuals with fasting plasma glucose (IFG) level 5.6 mmol/L) were classified as having impaired fasting glucose. Both IFG and IGT subjects were grouped together. Subjects with 2-hours glucose >11.1 mmol/L were considered as having type 2 diabetic mellitus (T2DM). NGT group was further divided into 2 sub-groups named NGT with a “favorable” metabolic risk profile (abbreviated NGT_fav) and NGT with an “unfavorable” metabolic risk profile (abbreviated NGT_Unfav) to take into account the hyperinsulinemia affecting subjects in the NGT group. Inclusion criteria for NGT subjects with a favorable risk profile (NGT_fav) were the following ones: BMI<25 for Caucasian and <23 for Asian subjects; waist <80 cm; waist/hip ratio <85; HOMA2-IR<1.21. IGT/IFG and T2DM subjects were also grouped together into a dysglycemic group. In total, 6 groups were reported: NGT_all; NGT_fav; NGT_unfav; IGT/IFG and; T2DM.

### Analytical Methods

Blood samples were collected into EDTA-containing tubes and spot urine samples were collected and frozen at −80° C. until analysis. For inositol isomers determination, analysis was performed on an Accela UHPLC (Thermo Fisher Scientific Inc., Waltham, Mass., USA) coupled to a TSQ Vantage triple quadrupole (Thermo Fisher Scientific Inc., Waltham, Mass., USA) equipped with a heated electrospray ionization (HESI) source in negative mode. Chromatographic separation was achieved using gradient elution on an UHPLC Acquity BEH Amide (1.7 μm, 2.1 mm×150 mm) column and guard column (Vanguard BEH Amide 1.7 μm, 2.1 mm×10 mm; Waters Corporation, Milford, Mass., USA) at 30° C. at a flow rate of 400 μL/min. Mobile phase consisted of 90% acetonitrile-NH₄OH 0.04% (eluent A) and 50% acetonitrile-NH₄OH 0.04% (eluent B). Column temperature was kept at 30° C. The injection volumes were 3 μL and 5 μL for urine and plasma respectively. Plasma samples were thawed and briefly vortexed before transferring 100 μL of the sample into a 1.5 mL microtube. 50 μL of [2H6]-myo-inositol, then 500 μL of ACN containing 1% of formic acid were added for protein precipitation. The tubes were vortexed for 1 min and centrifuged at 10,000×g for 3 min. Supernatants were transferred into an SPE Ostro 96 well plate for phospholipid removal. Positive pressure (about 8 psi) was applied for elution and the filtrates were then evaporate to dryness in a vacuum centrifuge (45° C., 5 mbar, 6 h). Dry residues were dissolved in 400 μL of water and transferred into a 0.45 μm centrifugal filter containing 175 mg of mixed bed ion exchange resin Amberlite MB-3. Tubes were spun at 3000×g for 2 min then the filtrates were evaporated to dryness in vacuum centrifuge at 45° C., 5 mbar for 2 h. Finally, residues were reconstituted in vials with 80 μL of ACN/water (60/40) and injected for analysis.

Urine samples were thawed and briefly vortexed before transferring 150 μL of sample into a 1.5-mL microtube. 100 μL of [2H6]-myo-inositol was added and the mixture was transferred into a 0.45 μm centrifugal filter containing 175 mg of mixed bed ion exchange resin Amberlite MB-3. Tubes were centrifuged at 3000×g for 2 min and the Amberlite was washed with 150 μL of water and eluted under centrifugation at 3000×g for 2 min. Filtrates were evaporated to dryness in a vacuum centrifuge at 45° C., 5 mbar for 2 h and the residues reconstituted in vials with 100 μL of ACN/water (60:40) were injected for analysis.

For glucose measures, blood samples were obtained in a Glucomedic tube (Greiner) as this is optimal for prevention of pre-test glycolysis. Raw laboratory glucose values were corrected for dilution by multiplying with 1.16 as per manufacturer's recommendation.

Homa2-IR was calculated using a computer model developed in 1996 and provides non-linear solutions using a downloaded software (www.OCDEM.ox.ac.uk). Subjects with non-haemolysed samples but with an insulin concentration lower than 2.88 uU/mL or 20 pmol/L, this minimum acceptable value by the software was used (n=10).

### Statistical Analysis

Statistical analysis was performed using R software (version 3.2.3). Mean and standard deviation were calculated. The statistical model used as covariate the glucose tolerance status, the ethnicity, the menstrual cycle, the age of the subjects and the BMI. For some statistical analysis some raw data were log transformed in view of their distribution. Pearson's correlations were also performed between inositol isomers and between inositol isomers and clinical outputs (i.e. anthropometric data and glucose and insulin levels). For the data imputation, values lower than the limit of quantification (LOQ) were replaced by half of the LOQ. LOQ was assessed at 25 nmol/L. P values lower than 0.05 were taken to be significant. In the different Tables, a letter represents significance at P<0.05 between NGT_fav and one other group (as pairwise comparison), whereas b letter represents significance at P<0.05 between NGT_all and one other group (as pairwise comparison) and c letter represents significance at P<0.05 between IGT/IFG and T2DM groups.

Results are shown in tables 1 to 7.

TABLE 1
Anthropometric profiles of withdrawn women from study 2

| | NGT | | | | |
|---|---|---|---|---|---|
| | (ALL) | | | | T2DM |
| | Mean | NGT_Fav | NGT_Unfav | IGT/IFG | Mean |
| | (SD) | Mean (SD) | Mean (SD) | Mean (SD) | (SD) |
| N | 182 | 63 | 119 | 41 | 26 |
| Age | 30.4 | 30.8 | 30.3 | 30.1 | 31.9 |
| | (3.7) | (3.4) | (3.9) | (4.2) | (4.9) |
| Anthropometric | | | | | |
| Weight (kg) | 67.67 | 54.45 | 74.7 | 74.4 | 74.25 |
| | (18.73) | (6.8) | (19.3)a | (23.3)a | (13.7)a |
| Height (cm) | 164.13 | 163.4 | 164.5 | 161.8 | 157.5 |
| | (6.8) | (6.20) | (7.1) | (6.3) | (6.2)a,c |
| BMI (kg/m2) | 25.05 | 20.4 | 27.6 | 28.4 | 29.9 |
| | (6.5) | (2.0) | (6.7)a | (8.5)a | (4.8)a |
| Waist (cm) | 83.3 | 71.0 | 89.9 | 88.9 | 93.9 |
| | (14.7) | (4.1) | (14.1)a | (18.3)a | (10.8)a |
| Hip (cm) | 98.99 | 89.4 | 104.1 | 103.5 | 104.2 |
| | (12.4) | (6.0) | (11.9)a | (16.5)a | (10.6)a |
| Menstrual cycle | | | | | |
| Regular, N (%) | 114 | 39 | 75 | 29 | 12 |
| | (62.6) | (61.9) | (63.0) | (70.7) | (46.2) |
| Irregular, N (%) | 68 | 24 | 44 | 12 | 14 |
| | (37.4) | (38.1) | (37.0) | (29.3) | (53.8) |
| Luteal phase | | | | | |
| Yes, N (%) | 44 | 11 | 33 | (11) | 8 |
| | (38.6) | (28.2) | (44.0) | (37.9 | (66.7) |
| No, N (%) | 70 | 28 | 42 | 18 | 4 |
| | (61.3) | (71.3) | (56.0) | (62.1) | (33.3) |

TABLE 2
Metabolic profiles of withdrawn women from study 2 by different glucose tolerance status

| | NGT (ALL) | NGT Fav | NGT Unfav | IGT/IFG | T2DM | Dysglycemic |
|---|---|---|---|---|---|---|
| | Mean (SD) | Mean (SD) | Mean (SD) | Mean (SD) | Mean (SD) | Mean (SD) |
| N | 182 | 63 | 119 | 41 | 26 | 67 |
| HOMA2-IR | 1.22 (1.03) | 0.68 (0.21) | 1.52 (1.16)^{a} | 2.04 (1.83)^{a,b} | 3.3 (2.17)^{a,b,c} | 2.54 (2.05)a,b |
| Glucose | | | | | | |
| (mmol/L) | | | | | | |
| Fasting | 4.97 (0.41) | 4.81 (0.33) | 5.06 (0.41) | 5.23 (0.51) | 7.03 (2.27)^{a,b,c} | 5.93 (1.70)a,b, |
| Post-30 min | 8.13 (1.46) | 8.02 (1.54) | 8.19 (1.42) | 9.76 (1.02)^{a,b} | 12.88 (3.35)^{a,b,c} | 10.96 (2.69)a,b |
| Post-120 min | 5.57 (1.13) | 5.42 (1.13) | 5.65 (1.13) | 8.46 (0.91)^{a,b} | 14.12 (3.78)^{a,b,c} | 10.65 (3.69)a,b |
| Insulin fasting | 9.53 (8.69) | 5.09 (1.98) | 11.88 (9.88)^{a,b} | 15.99 (15.01)^{a,b} | 24.88 (16.34)^{a,b,c} | 19.44 (16.03)a,b, |
| (pmol) | | | | | | |

TABLE 3
Plasma inositol isomers levels (μmol/L) by glucose tolerance status

| | NGT (all) | NGT_Fav | NGT_Unfav | IGT/IFG | T2DM |
|---|---|---|---|---|---|
| | Mean | Mean | Mean | Mean | Mean |
| | (SD) | (SD) | (SD) | (SD) | (SD) |
| Chiro- | 0.23 | 0.26 | 0.22 | 0.32 | 0.22 |
| inositol | (0.47) | (0.62) | (0.38) | (0.97) | (0.43) |
| Myo- | 25.91 | 25.44 | 26.16 | 23.23 | 24.66 |
| inositol | (5.9) | (5.8) | (5.9) | (4.8)* | (5.7) |
| Scyllo- | 0.67 | 0.72 | 0.64 | 0.46 | 0.37 |
| inositol | (0.43) | (0.48) | (0.40) | (0.30) | (0.23) |
| Myo-chiro | 470.6 | 509.5 | 450.1 | 540.2 | 447.5 |
| ratio | (608.7) | (804.7) | (476.2) | (411.3) | (465.4) |
| Myo-scyllo | 56.04 | 45.88 | 61.41 | 74.8 | 132.51 |
| ratio | (46.2) | (22.9) | (53.9) | (74.0) | (213.6) ^{a,b,*} |

TABLE 4
Characteristics of women with different glucose tolerance status

| | NGT (all) | NGT_Fav | NGT_Unfav | IGT/IFG | T2DM |
|---|---|---|---|---|---|
| | Mean (SD) | Mean (SD) | Mean (SD) | Mean (SD) | Mean (SD) |
| N | 182 | 63 | 119 | 41 | 26 |
| Age | 30.4 | 30.8 | 30.3 | 30.1 | 31.9 |
| | (3.7) | (3.4) | (3.9) | (4.2) | (4.9) |
| Weight | 67.67 | 54.45 | 74.7 | 74.4 | 74.25 |
| (kg) | (18.73) | (6.8) | (19.3) | (23.3) | (13.7) |
| Height | 164.13 | 163.4 | 164.5 | 161.8 | 157.5 |
| (cm) | (6.8) | (6.20) | (7.1) | (6.3) | (6.2) |
| BMI | 25.05 | 20.4 | 27.6 | 28.4 | 29.9 |
| (kg/m²) | (6.5) | (2.0) | (6.7) | (8.5) | (4.8) |
| Waist | 83.3 | 71.0 | 89.9 | 88.9 | 93.9 |
| (cm) | (14.7) | (4.1) | (14.1) | (18.3) | (10.8) |
| Hip | 98.99 | 89.4 | 104.1 | 103.5 | 104.2 |
| (cm) | (12.4) | (6.0) | (11.9) | (16.5) | (10.6) |
| HOMA2- | 1.22 | 0.68 | 1.52 | 2.04 | 3.3 |
| IR | (1.03) | (0.21) | (1.16) | (1.83) | (2.17) |
| Menstrual | | | | | |
| cycle | | | | | |
| Regular, | 114 | 39 | 75 | 29 | 12 |
| N (%) | (62.6) | (61.9) | (63.0) | (70.7) | (46.2) |
| Irregular, | 68 | 24 | 44 | 12 | 14 |
| N (%) | (37.4) | (38.1) | (37.0) | (29.3) | (53.8) |
| Luteal | | | | | |
| phase | | | | | |
| Yes, | 44 | 11 | 33 | 11 | 8 |
| N (%) | (38.6) | (28.2) | (44.0) | (37.9) | (66.7) |
| No, | 70 | 28 | 42 | 18 | 4 |
| N (%) | (61.3) | (71.3) | (56.0) | (62.1) | (33.3) |

TABLE 5
OGTT parameters by glucose tolerance status

| | NGT (all) | NGT_Fav | NGT_Unfav | IGT/IFG | T2DM |
|---|---|---|---|---|---|
| | Mean (SD) | Mean (SD) | Mean (SD) | Mean (SD) | Mean (SD) |
| N | 182 | 63 | 119 | 41 | 26 |
| HOMA2- | 1.22 | 0.68 | 1.52 | 2.04 | 3.3 |
| IR | (1.03) | (0.21) | (1.16)^{a} | (1.83)^{a,b} | (2.17)^{a,b,c} |
| OGTT- | | | | | |
| gluc. | | | | | |
| (mmol/L) | | | | | |
| Fasting | 4.97 | 4.81 | 5.06 | 5.23 | 7.03 |
| | (0.41) | (0.33) | (0.41) | (0.51) | (2.27)^{a,b,c} |
| Post-30 | 8.13 | 8.02 | 8.19 | 9.76 | 12.88 |
| min | (1.46) | (1.54) | (1.42) | (1.02)^{a,b} | (3.35)^{a,b,c} |
| Post-120 | 5.57 | 5.42 | 5.65 | 8.46 | 14.12 |
| min | (1.13) | (1.13) | (1.13) | (0.91)^{a,b} | (3.78)^{a,b,c} |
| Insulin | 9.53 | 5.09 | 11.88 | 15.99 | 24.88 |
| (pmol) | (8.69) | (1.98) | (9.88)^{a,b} | (15.01)^{a,b} | (16.34)^{a,b,c} |

TABLE 6
Plasma inositol isomers levels (μmol/L) by glucose tolerance status

| | NGT (all) | NGT_Fav | NGT_Unfav | IGT/IFG | T2DM |
|---|---|---|---|---|---|
| | Mean (SD) | Mean (SD) | Mean (SD) | Mean (SD) | Mean (SD) |
| Chiro- | 0.23 | 0.26 | 0.22 | 0.32 | 0.22 |
| inositol | (0.47) | (0.62) | (0.38) | (0.97) | (0.43) |
| Myo- | 25.91 | 25.44 | 26.16 | 23.23 | 24.66 |
| inositol | (5.9) | (5.8) | (5.9) | (4.8)* | (5.7) |
| Scyllo- | 0.67 | 0.72 | 0.64 | 0.46 | 0.37 |
| inositol | (0.43) | (0.48) | (0.40) | (0.30) | (0.23) |
| Myo-chiro | 470.6 | 509.5 | 450.1 | 540.2 | 447.5 |
| ratio | (608.7) | (804.7) | (476.2) | (411.3) | (465.4) |
| Myo-scyllo | 56.04 | 45.88 | 61.41 | 74.8 | 132.51 |
| ratio | (46.2) | (22.9) | (53.9) | (74.0) | (213.6) ^{a,b,*} |

TABLE 6a
Plasma myo-inositol: scyllo-inositol ratio by glucose
tolerance status by ethnic group

| | | | | | Dys- |
|---|---|---|---|---|---|
| myo/scyllo | NGT_fav | NGT_unfav | IGT/IFG | T2DM | glycemic |
| All, N = 249 | 45.9/1 | 61.4/1 | 74.8/1 | 132.5/1 | 97.2/1 |
| Asian, N = 144 | 55/1 | 79.1/1 | 84.2/1 | 135.1/1 | 107.8/1 |
| Caucasian, | 34.2/1 | 45.7/1 | 49.4/1 | NA | NA |
| N = 90 | | | | | |

TABLE 7
Urinary inositol isomers excretion (μmol/mmol of creatinine) by
glucose tolerance status

| | NGT (all) | NGT_Fav | NGT_Unfav | IGT/IFG | T2DM |
|---|---|---|---|---|---|
| | Mean (SD) | Mean (SD) | Mean (SD) | Mean (SD) | Mean (SD) |
| Creatinine | 11535 | 9552 | 12594 | 15790 | 12049 |
| (μmol/L) | (8729) | (7574) | (9142)^{a} | (9220)^{a,b} | (6287) |
| Chiro- | 0.24 | 0.26 | 0.22 | 0.20 | 0.42 |
| inositol | (0.56) | (0.51) | (0.57) | (0.55)* | (1.2) |
| Myo- | 9.21 | 10.86 | 8.33 | 6.35 | 17.97 |
| inositol | (11.68) | (15.0) | (9.37)* | (4.73)^{a} | (21.2)^{b} |
| Scyllo- | 5.04 | 6.34 | 4.34 | 3.31 | 3.36 |
| inositol | (3.58) | (5.26) | (1.93)^{a} | (1.69)^{a,b} | (1.16)* |
| Myo/chiro | 112.17 | 113.5 | 111.46 | 123.89 | 146.57 |
| ratio | (119.77) | (156.25) | (95.54) | (105.60) | (169.66) |
| Myo/scyllo | 1.86 | 1.64 | 1.98 | 2.13 | 5.44 |
| ratio | (1.40) | (1.14) | (1.51) | (1.31) | (5.87)^{a,b} |

TABLE 8
Plasma myo-inositol to scyllo-inositol ratio for subjects having a favourable glucose tolerance by ethnic group

| | e.groupWAO | N | P_MyoDIVscyllo | sd | median | q1.25% | q3.75% | min | max | se | ci |
|---|---|---|---|---|---|---|---|---|---|---|---|
| 1 | White | 23 | 34.23478 | 14.939136 | 29.630 | 26.3600 | 40.0000 | 13.94 | 75.75 | 3.115025 | 6.460167 |
| 2 | Asian | 36 | 55.02250 | 24.094572 | 52.025 | 35.7025 | 65.4275 | 15.39 | 127.78 | 4.015762 | 8.152430 |
| 3 | Other | 4 | 30.52000 | 5.930441 | 28.720 | 27.3600 | 31.8800 | 25.56 | 39.08 | 2.965221 | 9.436655 |

TABLE 9
Plasma myo-inositol to scyllo-inositol ratio for subjects having an unfavourable glucose tolerance by ethnic group

| | e.groupWAO | N | P_MyoDIVscyllo | sd | median | q1.25% | q3.75% | min | max | se | ci |
|---|---|---|---|---|---|---|---|---|---|---|---|
| 1 | White | 58 | 45.71431 | 30.25357 | 36.325 | 26.5325 | 52.960 | 16.40 | 170.86 | 3.972488 | 7.95477 |
| 2 | Asian | 54 | 79.10981 | 69.15278 | 56.920 | 37.4200 | 89.945 | 20.64 | 405.94 | 9.410501 | 18.87507 |
| 3 | Other | 7 | 54.85286 | 32.14697 | 45.690 | 35.9650 | 63.600 | 22.30 | 116.85 | 12.150411 | 29.73099 |

### Example 2

An example composition comprising scyllo-inositol and myo-inositol is set out in table 10.

The composition in table 10 may for example be a maternal supplement encapsulated in a soft gel capsule.

TABLE 10
Composition of Example 2

| Ingredient | Amount per daily dose | |
|---|---|---|
| Myo-inositol | 4 | g |
| Scyllo-inositol | 0.5 | g |

### Examples 3

An example composition comprising vitamin B2 in combination with myo-inositol, vitamins B6, B12 and D, and Lactobacillus rhamnosus GG¹ and Bifidobacterium lactis BB12² is set out in table 11.

The composition in table 9 may be a nutritional supplement in a powder form, intended to be sprinkled on food.

TABLE 11
Composition of Example 3

| Ingredient | Amount per daily dose | |
|---|---|---|
| Myo-inositol | 4 | g |
| Scyllo-inositol | 0.5 | g |
| Vitamin D | 10 | μg |
| Vitamin B6 | 2.6 | mg |
| Vitamin B12 | 5.2 | μg |
| Vitamin B2 | 1.8 | mg |
| Zinc | 10 | mg |
| β-carotene | 720 | μg |
| Folic acid | 400 | μg |
| Iron | 12 | μg |
| Calcium | 150 | μg |
| Iodine | 150 | μg |

| | |
|---|---|
| Lactobacillus rhamnosus GG¹⁾ | 1 × 10⁹ cfu |
| Bifidobacterium lactis BB12²⁾ | 1 × 10⁹ cfu |

¹⁾Strain deposited as CGMCC 1.3724
²⁾Strain deposited as CNCM I-3446

The composition may be provided as a kit of parts comprising in one sachet the probiotic as a powder and in a second sachet all other ingredients.

1. A method for use in the prevention or treatment of a condition selected from the group consisting of prediabetes, type II diabetes, gestational diabetes mellitus and a condition associated with any of the forgoing in a subject comprising administering a composition comprising a combination of scyllo-inositol and myo-inositol wherein, the scyllo-inositol and myo-inositol are nutritional ingredients to a subject in need of same.

2. A method according to claim 1, wherein the subject is a mammal selected from the group consisting of a cat, a dog and a human.

3. A method according to claim 1, wherein the Scyllo-inositol and myo-inositol are administered in a weight ratio of myo-inositol to scyllo-inositol in the range of 1:1 to 55:1.

4. A method according to claim 1, wherein the condition associated with prediabetes or type II diabetes is selected from the group consisting of cardio vascular disease, stroke, circulatory problems, diabetic retinopathy, kidney failure, hearing loss, fatty liver disease, an impaired cognitive ability, polycystic ovary syndrome and metabolic syndrome.

5. A method according to claim 1, wherein the condition associated with gestational diabetes mellitus is selected from the group consisting of preterm and caesarean delivery, birth injury to the mother or baby, shoulder dystocia, macrosomia, excessive offspring blood glucose concentration, excess weigh/adiposity and associated metabolic disorders.

6. A method according to claim 1, wherein the subject is at risk of suffering from pre-diabetes, type II diabetes or gestational diabetes mellitus.

7. A combination of scyllo inositol and myo inositol for use method according to claim 1, wherein the condition is gestational diabetes mellitus or a condition associated therewith and the subject is a woman who is trying to get pregnant, is pregnant or who is lactating or her offspring.

8. A method according to claim 1, wherein the scyllo-inositol is administered to a subject in an amount up to 1 g per day.

9. A method according to claim 1, wherein the myo-inositol is administered to a subject in an amount up to 5 g per day.

10. A method according to claim 1, wherein the scyllo-inositol and myo-inositol are administered in combination with a probiotic selected from the group consisting of Lactobacillus rhamnosus GG (CGMCC 1.3724), Bifidobacterium lactus BB-12 (CNCM I-3446), and a combination thereof.

11. A method according to claim 1, wherein the scyllo-inositol and myo-inositol are administered in combination with a an ingredient selected from the group consisting of: vitamin B2, vitamin B6, vitamin B12, vitamin D, iron, zinc and a combination of any of the foregoing.

12. A method according to claim 1, wherein the scyllo-inositol and myo-inositol are administered in combination with D-chiro inositol.

13. A method according to claim 1, wherein the scyllo-inositol and myo-inositol are administered in the form of a composition wherein, said composition is a product selected from the group consisting of: a nutritional product, a food product, a functional food product, a healthy ageing product, a dairy product, a nutritional supplement, a beverage product, a diet product, and a pet food product.

14. A method according to claim 13 wherein, the composition comprises myo-inositol and scyllo-inositol in a weight ratio of less than 80:1.

15. (canceled)
